(19) United States
(12) Patent Application Publication
(10) Pub. No.: US 2018/0236020 A1
(43) Pub. Date: Aug. 23, 2018

(54) BUCKWHEAT HONEY AND POVIDONE-IODINE WOUND HEALING DRESSING

(71) Applicant: SAN MELIX LABORATORIES, INC., PLANTATION, FL (US)

(72) Inventors: KENNETH A. SABACINSKI, PLANTATION, FL (US); JOHN L. KAUFMAN, POMPANO BEACH, FL (US)

(21) Appl. No.: 15/684,440

(22) Filed: Aug. 23, 2017

Related U.S. Application Data
(60) Provisional application No. 62/462,024, filed on Feb. 22, 2017.

(51) Int. Cl. A61K 36/69; A61K 9/00; A61K 35/644; A61K 31/79; A61K 9/06; A61L 26/00; A61K 33/40; A61K 31/7034

(52) U.S. Cl. CPC A61K 36/69; A61L 2300/412; A61K 35/644; A61K 31/79; A61K 9/06; A61L 26/0066; A61K 33/40; A61K 31/7034; A61L 26/008; A61L 26/0085; A61L 26/0052; A61L 2300/204; A61L 2300/30; A61L 2300/406; A61L 2300/41; A61K 9/0014

(57) ABSTRACT

The present invention is a method of treating acute and chronic wounds and skin conditions. The product comprises a composition or formulation comprising a mixture of buckwheat honey and povidone-iodine. In one unique embodiment the composition is gelled. The composition is applied directly to a wound or a patient's skin or is impregnated on gauze or other similar material on a bandage or dressing for application to an exuding or non-exuding acute or chronic wound or skin condition.

### CROSS-REFERENCE TO RELATED PATENT APPLICATION

This patent application is based upon and claims the benefit of the filing date of U.S. provisional patent application Ser. No. 62/462,024, filed Feb. 22, 2017, which is incorporated herein in its entirety.

### FIELD OF THE INVENTION

The invention herein relates to the use of buckwheat honey combined with one or more antibacterial compounds for the treatment of wounds and skin conditions. More particularly, buckwheat honey can be combined with povidone-iodine, optionally with other components, to provide a potent wound-healing and/or skin care therapy with unique healing qualities.

### BACKGROUND OF THE INVENTION

Honey has been used as a medicinal aid since ancient times and has been well established in its ability to successfully treat acute and chronic wounds. Honey has been found to be effective in reducing infection and aiding healing. Honey obtained predominately from one flower source is considered monofloral. Individual honeys obtained from a single flowering plant species (monofloral honeys) have specific characteristics, which allow a monofloral honey to be utilized as a therapeutic substance. Monofloral honeys also have a high level of antioxidants, which helps prevent the cell damage caused by reactive oxygen species and have been well documented to have excellent broad spectrum antibacterial and antifungal properties. As a result of these findings, numerous studies have been conducted to understand the efficacy of monofloral buckwheat honey in treating acute and chronic wounds.

Buckwheat honey has been well known in the holistic medicine world as a medicinal aid because it has a high mineral content, antioxidant compounds, and healing properties. Physically, buckwheat honey is characterized as having a dark brown color with a red tint and is also known for a unique, woody aroma. Chemically, buckwheat honey is a complex mixture of fructose, glucose, sucrose, and water, and it particularly has higher concentrations of macronutrients, various trace elements, and antioxidant compounds. To create this complex mixture, honeybees collect nectar, and through digestion the honeybees add unique digestive proteins, which give the honey its unique healing characteristics.

Various honeys have been shown to be safe and an effective dressing for skin ulcerations, including Wagner grade-II diabetic foot ulcers. Additionally, dressings using honey have shown decreased wound edema and reduced odor more effectively than non-honey dressings. Buckwheat honey provides characteristics that provide effective and broad spectrum antibacterial activity and antifungal properties. See, FIGS. 1 to 4, where the antibacterial effectiveness of each of willow herb honey, heather honey, buckwheat honey, lingonberry honey, and cloudberry honey against Staphylococcus aureus, MRSA, Streptococcus pneumoniae, and Streptococcus pyogenes, respectively, is shown. Huttunen S, Riihinen K, Kauhanen J, Tikkanen-Kaukanen, C. Antimicrobial activity of different mono floral honeys against human pathogenic bacteria. APMIS: acta pathologica, microbiologica, et immunological Scondinavica. September, 2013, 121(9): 827-834. In addition, buckwheat honey has a higher concentration of polyphenols and H₂O₂ (hydrogen peroxide) and has other healing properties.

Brudzynski, K, et al., Powerful bacterial killing by buckwheat honeys is concentration-dependent, involves complete DNA degradation and requires hydrogen peroxide. Frontiers in Microbiology July 2012; Vol 3, Article 242, reported the effectiveness of buckwheat honey's antibacterial effect through its high concentration of hydrogen peroxide. The study reported that buckwheat honey uses the bactericidal effects of hydroxyl radicals while maintaining an antioxidant concentration sufficiently high to prevent cellular damage. By implementing this very unique mechanism, buckwheat honey is able to kill bacteria while protecting bodily tissues from the oxidative stress used to kill the microorganisms.

In terms of application, a study reported in Van den Berg et al., An in vitro examination of the antioxidant and anti-inflammatory properties of buckwheat honey. Journal of Wound Care, Vol. 17, No. 4, April 2008, pp. 172-178, looked at the effectiveness of different types of honey on wound healing. The study considered the different ways that honey could promote healing in injured tissue. After compiling all of the data, the authors found that buckwheat honey was the most effective wound-healing honey.

In an article discussing the mechanism of how honey induces wound repair, it was shown that buckwheat honey has an ability to promote cellular regeneration superior to other healing remedies, specifically manuka honey. Ranzato E, Martinotti S, Burlando B. Epithelial mesenchymal transition traits in honey-driven keratinocyte wound healing: Comparison among different honeys. Wound Repair Regen. 2012; 20:778-85. doi: 10.1111/j.1524-475X.2012.00825.x. [PubMed].

In a Finnish study, five different honeys were used in concentrations of 60%, 40%, and 20% by weight in phosphate buffered saline. The highest antimicrobial activity was recorded when the honey had a concentration of 40% or 60% by weight, but significant antimicrobial activity was also detected even when the honey was at 20% concentration by weight. Sanna Uttunen, et al., “Antimicrobial activity of different Finnish monofloral honeys against human pathogenic bacteria.” ACTA Pathologica Microbiologica Et Immunologica Scandinavica, 2012, pages 827-834.

Honey has been used in clinical practice for many types of diseases since ancient times. It is still being used because it enhances auto-debridement by absorbing edematous fluid due to its high osmolarity. Van den Berg et al. data demonstrated that honey exposure induces low cytotoxicity on fibroblasts. This finding confirms previous results on keratinocytes' increased ability to regenerate epithelial cells, suggesting that honey can be considered a safe compound for external applications. Scratch wound data and cell migration assay showed that honey improves fibroblast wound repair capabilities. Cell motility is a key element of tissue repair processes. Therefore, its increased induction could explain the ability of honey to promote the activation of fibroblasts at a faster rate, similar to what was reported for keratinocytes.

To understand the mechanisms underlying this effect, i.e., promoting the activation of fibroblasts at a faster rate, various studies used a battery of inhibitors of cell signaling pathways that are known to be directly involved in the wound healing process. BAPTA-AM (1,2-bis(2-aminophenoxy)ethane-N,N,N′,N′-tetraacetic acid tetrakis(acetoxymethyl ester)) was found to be the most effective inhibitor of scratch wound closure, showing the essential role of intracellular calcium in the wound-healing process. Current literature provides evidence that matrix metalloproteinases (MMPs) and their inhibitors are essential in tissue repair. MMPs and tissue inhibitors of metalloproteinases (TIMPs) are known to control the inflammatory phase of wound healing by modulating chemokine and cytokine activation. They also regulate matrix degradation and remodeling, which is an essential step in wound repair. Majtan and Ranzato have consistently demonstrated that honey exposure induces gelatinase MMP-9 expression in human keratinocytes, which confirms a similar mechanism already observed on platelet lysate added cells. In contrast, the platelet lysate does not seem to affect gelatinase expression in fibroblasts. Ranzato E, Martinotti S, Volante A, Mazzucco L, Burlando B. Platelet lysate modulates MMP-2 and MMP-9 expression, matrix deposition and cell-to-matrix adhesion in keratinocytes and fibroblasts. Exp Dermatol. 2011; 20:308-13. doi: 10.1111/j.1600-0625.2010.01173.x. [PubMed]. This is another point of convergence with the present investigation, where MMP or TIMP upregulation by honey was limited to MMP-3 induction with manuka, and TIMP-1 with manuka and buckwheat. The increase of TIMP-1 upon buckwheat exposure could be linked to the anti-inflammatory effects of this protein, considering that it has been induced by cytokine exposure in dermal fibroblasts.

Studies consistently indicate that honey is generally active in facilitating wound closure or healing, thus confirming much anecdotal and scientific evidence. However, a key finding is that different types of honey work through different mechanisms, and some of these mechanisms are more efficient and effective than others. In particular, buckwheat honey has shown more powerful wound-healing properties than manuka honey on both dermal and epidermal cells. Hence, the complex of different honey types offers a wider set of therapeutic possibilities, raising new medical interest in this valuable natural product. See, Elia Ranzato, et al., “Honey exposure stimulates wound repair of human dermal fibroblasts.” Burns & Trauma, June 2013, Vol 1, Issue 1. It is nature's invention of a natural wound healing dressing. When buckwheat honey is combined with povidone-iodine, results are significantly improved.

Povidone-iodine (PVP-I, polyvinylpyrolidone), also known as iodopovidone, is an antiseptic used for skin disinfection before and after surgery. It was developed for commercial use in 1955. Povidone-iodine is on the World Health Organization (WHO) list of essential medicines. Since povidone-iodine kills microorganisms, it may be used to disinfect both the skin of a patient and the hands of healthcare providers.

Povidone-iodine is antibacterial, antifungal, antiprotozoal, and antiviral. Povidone-iodine can be loaded into hydrogels, carboxyethyl cellulose (CMC), polyvinyl alcohol (PVA), gelatin, and crosslinked polyacrylamide. The rate of release of the iodine is dependent upon the hydrogel composition. When povidone-iodine is used with CMC/PVA, there is a higher rate of release. When povidone-iodine is used with a greater amount of gelatin, there is a slower release, which is effective for diaper rash, dermatitis, Tinea, and athlete's foot infection. Povidone-iodine may also be used for minor wounds as a dressing and may be applied to the skin as a liquid, gel, cream, or ointment.

### OBJECTS OF THE INVENTION

It is an object of the invention to provide a wound-healing or skin condition treatment formulation or composition comprised of buckwheat honey and one or more bacterial agents.

It is a further object of the invention to provide a wound-healing or skin condition treatment formulation or composition which comprises povidone-iodine as an antibacterial agent.

It is a yet further object of the invention to provide a wound-healing or skin condition treatment formulation or composition wherein the povidone-iodine comprises an about 9 to about 12% by volume iodine solution mixed with sterile water to dilute it to from about 1.5 to about 4.0% by volume, preferably about 2.5%.

It is a yet further object of the invention to provide a wound-healing or skin condition treatment formulation or composition wherein the povidone-iodine is in the form of a white powder.

It is a yet further object of the invention to provide a wound-healing or skin condition treatment formulation or composition which can be provided in a tube or spray.

It is a yet further object of the invention to provide a wound-healing or skin condition treatment dressing comprising substrate material and an effective amount of a wound-healing or skin condition treatment formulation comprised of buckwheat honey and one or more bacterial agents.

It is a yet further object of the invention to provide a wound-healing or skin condition treatment dressing wherein wound-healing or skin condition treatment formulation or composition is impregnated into a reinforcing fiber substrate or bandage for application to a wound or a patient's skin and can be released from the reinforcing fiber substrate.

It is a yet further object of the invention to provide a method of treating a wound or skin condition on or in a patient which comprises directly applying to the wound or the patient's skin an effective amount of a wound-healing or skin condition treatment formulation or composition comprised of buckwheat honey and one or more bacterial agents.

It is a yet further object of the invention to provide a method of treating a wound or skin condition on a patient which comprises contacting the wound or the patient's skin with a wound-healing or skin condition treatment dressing comprising substrate material and an effective amount of wound-healing or skin condition treatment formulation or composition comprised of buckwheat honey and one or more bacterial agents.

These and other objects of the invention will become more apparent from the discussion below.

### SUMMARY OF THE INVENTION

The novel and unique combination of buckwheat honey and an antibacterial agent such as povidone-iodine offers overlapping benefits in wound treatment or in treating skin conditions. The invention herein relates to the specific use of a composition or formulation comprising buckwheat honey with one or more antibacterial compounds such as povidone-iodine for use directly on wounds or on a patient's skin, or on or in a dressing applied to a wound or a patient's skin. The composition can be impregnated on a bandage or dressing substrate such as acetate, cotton, or other material useful to carry the composition. When used directly on a wound or a patient's skin, the formulation or composition preferably includes a gelling agent that thickens the composition.

The combination of buckwheat honey and povidone-iodine as an antibacterial formulation or composition is a preferred embodiment that provides a potent wound care therapy or skin condition treatment with unique healing qualities. It offers broad spectrum antibacterial activity along with antifungal and antiviral properties. This combination gives antibacterial effects that are effective on, for example, methicillin resistant Staphylococcus aureus. Each gram of honey will have a povidone-iodine concentration in a narrow range of from about 0.1 to about 5% weight per volume (“w/v”), that is, from about 0.1 to about 5 grams of povidone-iodine for every 100 ml of honey. This range will augment the antibacterial properties of the buckwheat honey. A preferred range for the concentration of povidone-iodine is about 2.5% w/v per 4 grams of buckwheat honey.

Potential uses include, but are not limited to, skin irritation, rashes, dermatitis, pruritus, fungal infection, diaper rash, minor skin infections, burns (minor and severe), ulcerations (superficial, deep, and to all depth levels), surgical wounds, traumatic wounds, gunshot wounds, puncture wounds, chemical burns, insect bites, animal bites, draining, and non-draining wounds, neuropathic and vascular wounds, decubitus wounds, and other skin conditions and acute and chronic wounds.

In an aspect of the invention, a therapeutic wound-healing or skin condition treatment formulation or composition comprises a wound-healing or skin condition treating effective amount of buckwheat honey and one or more antibacterial agents.

In another aspect of the invention, a therapeutic wound-healing or skin condition treatment formulation or composition comprises a wound-healing or skin condition treating effective amount of buckwheat honey and povidone-iodine.

In a further aspect of the invention, in a wound-healing or skin condition treatment formulation or composition, the buckwheat honey is 100% pure and strained.

In a further aspect of the invention, a wound-healing or skin condition treatment formulation or composition is applied directly to a wound or the patient's skin.

In a further aspect of the invention, in a wound-healing or skin condition treatment formulation or composition, povidone-iodine is present in an amount of from about 1 to about 5% w/v to about 4 grams of buckwheat honey.

In a further aspect of the invention, in a wound-healing or skin condition treatment formulation or composition, povidone-iodine is present in an amount of from about 2.5% w/v to about 4 grams of buckwheat honey.

In a further aspect of the invention, a wound-healing or skin condition treatment formulation or composition includes a gelling agent.

It is a yet further aspect of the invention to provide a wound-healing or skin condition treatment formulation or composition wherein the gelling agent is sufficient to thicken the mixture and to substantially prevent the formulation or composition from running and to release a desired amount of the formulation or composition to the wound or skin condition.

In a further aspect of the invention, a wound-healing or skin condition treatment formulation or composition can be provided in a tube or spray.

In a further aspect of the invention, a wound-healing or skin condition treatment dressing comprises a bandage or substrate material and an effective amount of a wound-healing or skin condition treatment formulation or composition comprised of buckwheat honey and one or more bacterial agents.

In a further aspect of the invention, a wound-healing or skin condition treatment dressing comprises a bandage or reinforcing fiber substrate material and a wound-healing or skin condition treatment formulation or composition comprised of buckwheat honey and povidone-iodine.

In a further aspect of the invention, a wound-healing or skin condition treatment bandage or dressing comprises an effective amount of the wound-healing or skin condition treatment formulation or composition.

In a further aspect of the invention, in a wound-healing or skin condition treatment dressing, the substrate material comprises acetate, cotton (medical gauze), or alginate.

In a further aspect of the invention, a wound-healing or skin condition treatment dressing comprises a 5″×4″ adhesive or non-adhesive bandage with from about 2 to about 8 grams of wound-healing or skin condition treatment formulation or composition.

In a further aspect of the invention, a wound-healing or skin condition treatment bandage or dressing comprises about four grams of wound-healing or skin condition treatment formulation or composition.

In a further aspect of the invention, in a wound-healing or skin condition treatment dressing, the wound-healing or skin condition treatment formulation or composition is impregnated into a bandage or reinforcing fiber substrate for application to a wound or a patient's skin and is released from the bandage or reinforcing fiber substrate.

In a further aspect of the invention, a method of treating a wound on or in a patient or a skin condition on a patient's skin comprises directly applying an effective amount of a wound-healing or skin condition treatment formulation or composition comprised of buckwheat honey and povidone-iodine.

In a further aspect of the invention, a method of treating a wound or skin condition on a patient comprises contacting the wound or skin condition with a wound-healing or skin condition treatment bandage or dressing comprising a reinforcing fiber substrate material and a wound-healing or skin condition treatment formulation or composition comprised of buckwheat honey and povidone-iodine.

In a further aspect of the invention, the buckwheat honey and povidone-iodine formulation or composition is applied in a cream.

In a further aspect of the invention, the buckwheat honey and povidone-iodine formulation or composition is applied in an ointment.

In a further aspect of the invention, in a wound-healing or skin condition treatment formulation or composition, povidone-iodine is present in an amount of from about 1 to about 5% w/v to about 2 to about 8 grams of buckwheat honey.

In a further aspect of the invention, in a wound-healing or skin condition treatment formulation or composition, povidone-iodine is present in an amount of from about 2.5% w/v to about 4 grams of buckwheat honey.

These and other advantages of the present invention will be readily appreciable from the following description of preferred embodiments of the invention and from the accompanying drawings and claims.

### BRIEF DESCRIPTION OF THE DRAWINGS

FIG. 1 is a graph representing the antibacterial effectiveness of buckwheat honey, heather honey, willow herb honey, lingonberry honey, and cloudberry honey, respectively, against Staphylococcus aureus;

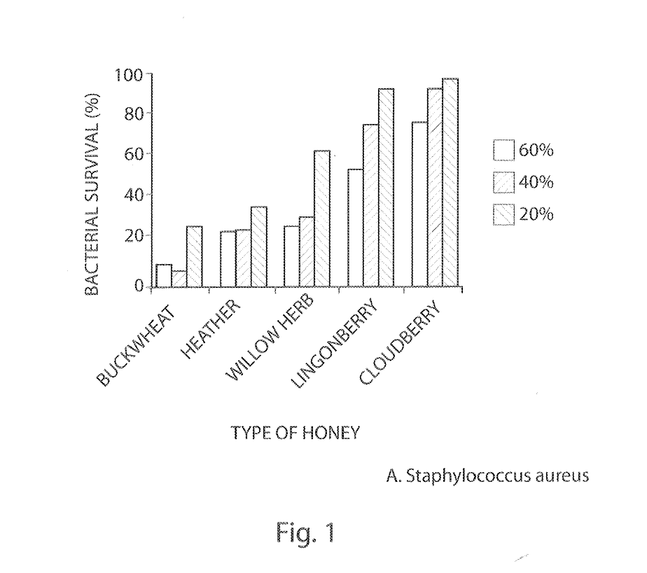

FIG. 2 is a graph representing the antibacterial effectiveness of buckwheat honey, heather honey, willow herb honey, lingonberry honey, and cloudberry honey, respectively, against MRSA;

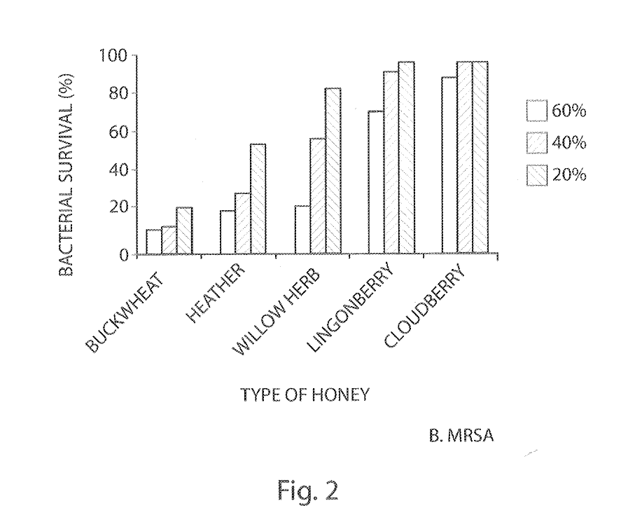

FIG. 3 is a graph representing the antibacterial effectiveness of buckwheat honey, heather honey, willow herb honey, lingonberry honey, and cloudberry honey, respectively, against Streptococcus pneumoniae; and

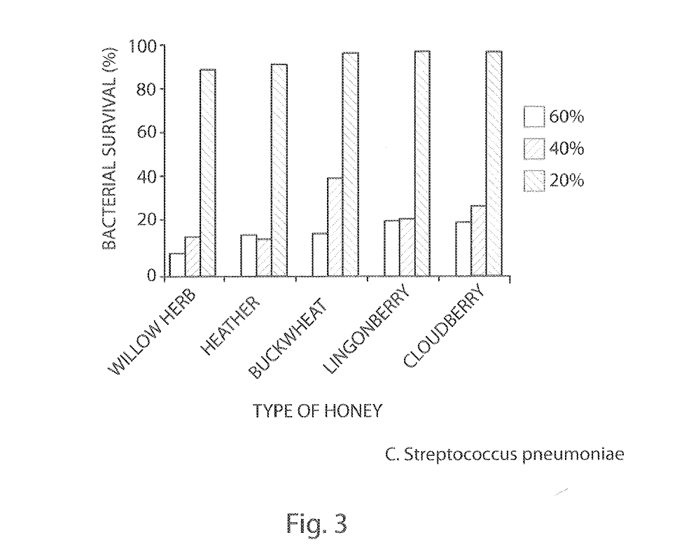

FIG. 4 is a graph representing the antibacterial effectiveness of buckwheat honey, heather honey, willow herb honey, lingonberry honey, and cloudberry honey, respectively, against Streptococcus pyogenes.

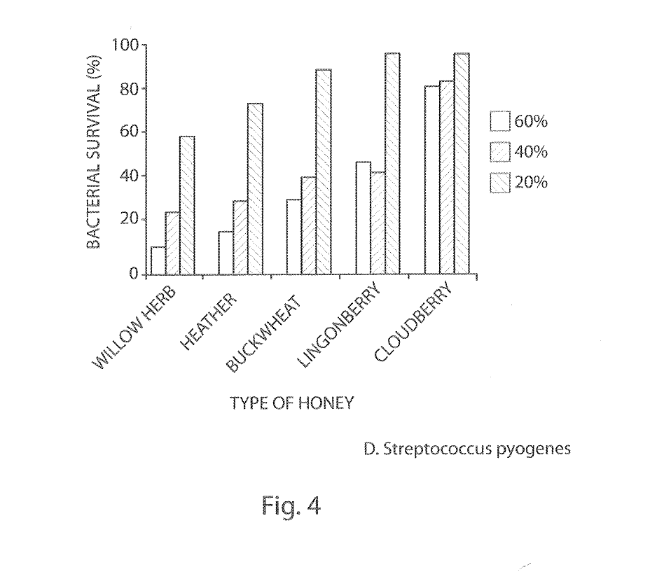

### DETAILED DESCRIPTION OF THE INVENTION

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

The term “wound-healing or skin condition treatment formulation or composition” as used herein refers to a formulation or composition designed to treat wounds, lesions, skin conditions, or injuries caused by physical, metabolic, or chemical means. The wound-healing or skin condition treatment process comprises a series of steps where injured tissue is repaired or regenerated. This usually involves three steps: (a) an inflammation stage (0-3 days), (b) a cellular proliferation phase (3-12 days), and (c) a remodeling phase (3-6 months).

Typically a wound-healing or skin condition treatment formulation or composition has at least one active ingredient that helps to repair a wound or treat a skin condition. Examples of active ingredients include, but are not limited to, povidone-iodine, methylglyoxal, bacitracin, neomycin, and polymyxin (e.g., polymyxin B sulfate). A wound-healing or skin condition treatment formulation or composition may also contain inactive ingredients that are typically required for formulation. (See, e.g., U.S. Pat. No. 5,652,274 and U.S. Published Patent Application No. 2005/0043253, each of which is incorporated herein by reference.)

Healing time related to wounds or skin conditions, whether acute or chronic, depends on multiple factors. These factors include, but are not limited to, patient medical history, etiology, location, size, depth, length of time present, previous treatment, and drainage (purulent, synovial, serous, and serosanguinous).

As discussed above, wound-healing or skin condition treatment normally progresses through different phases. Wounds and skin conditions will heal in days to months. Healing may be delayed for any number of reasons. Some of these reasons include, but are not limited to, poor circulation, infection, elevated protease levels, pressure (weight-bearing), increased motion, patient non-compliance, and the like.

A “chronic wound,” “non-healing wound,” slow-to-heal wound,” or “stalled wound,” as used herein, refers to a wound that fails to heal over a 4 to 12 week timeframe from inception of the wound to complete closure of the skin at the wound site. Such wounds commonly include external dermal wounds.

Skin wounds designated as “chronic”, “non-healing”, “slow-to-heal”, or “stalled” are commonly observed in clinical settings as venous leg ulcers, diabetic foot ulcers, pressure ulcers, arterial ulcers, ulcers of mixed etiology, burns, or non-healing surgical wounds. Other types of non-healing wounds or conditions are observed in less frequent conditions, such as, fistulae, dermatitis or vasculitis wounds, skin cancers, and radiation burns. This list is not exhaustive and is provided to show examples of such non-healing wounds or skin conditions. These wounds or skin conditions are differentiated from “acute” wounds or skin conditions that spontaneously heal without complications in a matter of days or weeks through the four normal phases of the “wound-healing curve” (hemostasis, inflammation, proliferation, and remodeling). Chronic wounds or skin conditions may persist for months or years and occasionally can last a lifetime and are therefore commonly referred to as “non-healing” wounds. There is a need for treatment of any of these types of non-healing wounds or skin conditions since spontaneous healing has failed to occur. In chronic wounds or skin conditions, at the cellular biological level, there is commonly a prolonged inflammatory phase often caused by elevated proteases or active infection.

Povidone-iodine was developed for commercial use in 1955. The Federal Drug Administration (FDA) has approved povidone use and stated that povidone is generally considered safe (GRAS). The Environmental Protection Agency (EPA) has concluded that iodine and iodophor complexes are very low toxicity by oral or dermal exposure.

Since it was developed, povidone-iodine has been used for the treatment of all types of wounds. Wounds such as vascular (arterial and venous), neuropathic, pressure, traumatic, post-operative, and infected wounds responded to treatment with povidone-iodine. In addition, treatment with povidone-iodine included wounds of varying depths ranging from pre-ulceration, superficial, and deep to ulcerations that extend to the tendons, joints, and bones.

In treatment with full strength (10% w/v) povidone-iodine, it was found to be too caustic and drying to the healing tissues. These issues with full strength (10% w/v) povidone-iodine led to accepted wisdom and numerous articles in the literature (for example, Kramer S, Effect of Povidone iodine on wound healing. J Vascular Nursing. 17(1):17-23. April 1999) to advocate against the use of full strength povidone-iodine (10% w/v) for wound healing.

During clinical treatment of all types and depths of wounds, treatment protocol was developed using a dilution of povidone-iodine. Various dilutions of povidone-iodine (from about 0.01 to about 10% w/v) were clinically tested over the past thirty years. The results showed that the current proposed accurate and effective dilution of about 2.5% w/v povidone-iodine has had the best reproducible effects on wound healing. The negative effects from full strength povidone-iodine (10%) were corrected by diluting the povidone-iodine to about 2.5% w/v solution.

There is no decrease in the antimicrobial properties of povidone-iodine at the about 2.5% dilution. In addition, there has been no bacterial resistance found or developed to povidone-iodine.

Further research and clinical case studies conducted on preventing and treating wounds, preventing chronicity of wounds, promoting antioxidation of healthy cells and tissue, and facilitating faster healing led to investigation of the combination of honey with povidone-iodine. Earlier studies used generic honey (sugar) and povidone-iodine in no specific combination or dilution. Buckwheat honey (fagopyrum esculentum) showed increased antioxidant, anti-inflammatory, and at least equal antibacterial properties as compared to other mono-floral honeys. The unique combination of buckwheat honey and povidone-iodine creates specific and unique benefits to wound healing. The buckwheat honey/povidone-iodine formulation or composition is for use in direct application or as part of a wound dressing for treatment of all types and depths of wounds and acute or chronic skin conditions or skin ulcerations regardless of vascular and neurologic status.

An embodiment of a buckwheat honey/povidone-iodine formulation or composition according to the invention comprises a medicinal honey with concentrations of hydrogen peroxide and polyphenols that are all in the upper concentration range of what are naturally found in various native honeys. In this first formulation, buckwheat honey provides a composition containing buckwheat honey with high natural concentrations of hydrogen peroxide and polyphenols to which povidone-iodine is added to a concentration between 500 mg/kg and 2,000 mg/kg. The phrase “high natural concentrations of hydrogen peroxide and polyphenols” is defined as concentrations that naturally exist in buckwheat honey where the concentration of hydrogen peroxide is in the range of about 2-4 mM and where the polyphenol concentration is in the range of about 275-575 gallic acid equivalents per gram of honey.

The buckwheat honey useful according to the invention is pure and strained, preferably seasonally harvested raw, monofloral buckwheat honey naturally rich in hydrogen peroxide (containing and/or capable of generating about 2-4 mM concentration of hydrogen peroxide), polyphenols (275-575 gallic acid equivalents of polyphenol compounds per gram of honey). Buckwheat honey, which is derived from the buckwheat flower, is dark brown with a red tint and typically has a pH of from about 3.0 to about 7.5. It has a shelf-life of two or more years if stored in a sealed container at from about 0° to 10° C. Useful buckwheat honey comprises medicinal grade, pure, unheated, microfiltered buckwheat honey such as is commercially available from, for example, Hackenberg Apiaries, Lewisburg, Pa., and other commercial apiaries.

Povidone-iodine is a natural antibacterial compound. It typically is a white powder with a pH of from about 1.5 to about 5.0, ideally about 4.5, when diluted in sterile water. Povidone-iodine has a shelf-life of about 12 months if stored in a light-resistant container at a temperature not to exceed about 35° C. Povidone-iodine is commercially available from, for example, Sigma-Aldrich, St. Louis, Mo.

A useful preparation comprising buckwheat honey and povidone-iodine comprises providone-iodine in a concentration of from about 1 to about 5% w/v povidone iodine, preferably about 2.5% w/v solution, of final honey product. This buckwheat honey/povidone-iodine composition has broad-spectrum antimicrobial activity effective to reduce the number of viable microorganisms at a wound site.

The buckwheat honey/povidone-iodine formulations or compositions are used in aqueous, ointment, or wound-dressing formulations to modulate biochemical mechanisms associated with wound healing, including decreasing both wound protease activities and active infection.

The buckwheat honey/povidone-iodine formulations or compositions can be applied directly to a wound or skin condition. In another aspect, the present invention provides a bandage or dressing for wound healing or treatment of a skin condition. The bandage or dressing comprises one or more of the buckwheat honey/povidone-iodine formulations or compositions described herein and a support or substrate. In any embodiment of the bandage or dressing, the support or substrate includes, but is not limited to, a fibrous gauze material, a hydrogel, a foam, a film, a hydrocolloid, an alginate, a collagen, or a combination of any two or more of the aforementioned. Useful bandages or dressings include BAND-AID® or similar bandages that comprise adhesive backings with cotton or other material that adheres to the adhesive backings and absorbs the buckwheat honey/povidone-iodine formulations or compositions.

In another aspect of the invention, a wound or skin condition is treated with a buckwheat honey/povidone-iodine formulation or composition described herein.

In another embodiment of the buckwheat honey/povidone-iodine formulation or composition, the various compositions are used prophylactically to prevent surgical wounds in high-risk patients from post-operative wound dehiscence and non-healing. More than 53 million people undergo surgical procedures annually in the United States, with about half of these occurring under general anesthesia. Post-operative wound dehiscence has been investigated in several studies, and a small incidence has shown to be an issue of concern in all age groups, including the pediatric population, but with a higher incidence in the older population. The failure of these surgical wounds to heal in a normal time frame pushes them into the category of chronic wounds. Diabetes, obesity, cancer therapy, and vascular abnormalities, which are all increasing in incidence in the Western population, contribute to delayed healing and are considered risk factors. The prophylactic application of one or more of the buckwheat honey/povidone-iodine formulations herein to surgical incisions post-operatively in “high-risk” patients will aid in reducing the incidence of non-healing surgical wounds.

The components of the buckwheat honey/povidone-iodine formulation or composition surprisingly provide a synergistic effect that results in the suppression of the accumulation of a biochemical marker (e.g., proteases, and pro-inflammatory cytokines) associated with inflammation, and the up-regulation of other biochemical markers (e.g. growth factors and protease inhibitors) associated with wound healing. In addition, the components of the buckwheat honey/povidone-iodine formulations or compositions provide antibacterial activity that acts concomitantly with the anti-inflammatory activity to also provide suppression of active infections.

In some aspects of the invention pH adjusters may be needed and used in an amount which produces the desired function provided the amount of the pH adjuster does not affect the stability of composition. These pH adjusters are acids or bases that can be used to adjust the pH of the finished composition or formulation to a desired level. Examples of useful pH adjusters include, but are not limited to, acetic acid, ammonia, citric acid, ethanolamine, formic acid, oxalic acid, potassium hydroxide, sodium hydroxide, and triethanolamine. The pH adjusters are used in an effective, pH-adjusting amount of, for example, from about 0.01 to about 2.0% by weight, preferably from about 0.05 to about 1.0% by weight, based upon the desired effect. If buffering agents are required, the compounds can be used to maintain a desired pH in an aqueous environment. Examples of such buffers include, but are not limited to, boric acid, citric acid, lactic acid, fumaric acid, phosphoric acid, and salts thereof. The buffering agents are used in an effective, buffering amount of, for example, from about 0.01 to about 2.0% by weight, preferably from about 0.05 to about 1.0% by weight, based upon the desired effect.

In another aspect of the invention, the buckwheat honey/povidone-iodine formulation or composition may additionally comprise one or more hemostatic agents. Examples of such hemostatic agents include, for example, extracellular matrix protein, kaolin, zeolite, chitosan, anhydrous aluminum sulfate, and aluminum chloride hexahydrate. The hemostatic agents are used in an effective, hemostatic amount of, for example, from about 0.01 to about 2.0% by weight, preferably from about 0.05 to about 1.0% by weight, based upon the desired effect.

In another aspect of the invention addition, the buckwheat honey/povidone-iodine formulation or composition may additionally compromise one or more anesthetic agents. Examples of such anesthetic agents include, but are not limited to, lidocaine 1% and 2%, with and without epinephrine, and bupivacaine 0.25% and 0.5%, with and without epinephrine. The anesthetic agents are used in an effective, anesthetic amount of, for example, from about 0.01 to about 2.0% by weight, preferably from about 0.05 to about 1.0% by weight, based upon the desired effect.

In another aspect of the invention, the buckwheat honey/povidone-iodine composition may additionally comprise one or more anesthetic agents and one or more hemostatic agents.

In another aspect of the invention, a wound-healing or skin condition treatment dressing includes a gelling agent to thicken the mixture and to substantially prevent the composition from running and to release a desired amount of the dressing to the wound. Examples of useful gelling agents include, but are not limited to, natural polymers-proteins like gelatin, casein, collagen, egg whites, polysaccharides and semi synthetic polymers-cellulose subordinates such as carboxylmethyl cellulose, ethylcellulose, hydroxyethyl cellulose, hydroxypropyl cellulose, magnesium aluminum silicate, methylcellulose, sodium alginate, etc. The gelling agents are used in an effective, gelling amount of, for example, from about 0.01 to about 2.0% by weight, preferably from about 0.05 to about 1.0% by weight, based upon the desired effect.

Buckwheat honey/povidone-iodine formulations or compositions according to the present invention are useful for treating common chronic wounds, such as venous leg ulcers, diabetic foot ulcers, pressure ulcers, arterial ulcers, burns, non-healing surgical wounds, chronic rhinosinusitis and metritis. In addition, such formulations or compositions are also useful for treating abrasions, lacerations, minor cuts, scalds and burns, and other partial thickness wounds. The formulations or compositions are advantageously applied in a cream or ointment base for a period of time (e.g., several hours to several days) that are optionally impregnated into or associated with carrier dressing supports (e.g., fibrous gauze, hydrogel, foam, film, hydrocolloid, collagen, or alginate).

The buckwheat honey/povidone-iodine formulation or composition can be applied directly to a wound as a cream or spray or administered on a bandage or dressing comprising a substrate. For example, a 5″×4″ dressing containing the buckwheat honey/povidone-iodine formulation or composition could contain approximately four grams of pure strained buckwheat honey and about 1 to about 5% w/v (preferably about 2.5% w/v) of povidone-iodine.

This “standard” formulation is useful applied to a substrate in variously sized bandages or dressings or in an ointment or cream from a tube or spray. This formulation will preferably be sterilized by gamma radiation. This sterilization process was chosen because gamma radiation (25 kGy) did not affect the properties of the buckwheat honey.

The present disclosure further provides a method for treating wounds or skin conditions. In some embodiments, the method includes contacting a wound or skin condition with any buckwheat honey/povidone-iodine formulation or composition disclosed herein wherein the composition includes a medicinal honey fortified with the natural non-peroxide antibacterial compound, povidone-iodine; a pharmaceutically acceptable carrier; 1-5% w/v and the natural non-peroxide antibacterial compound effective to reduce the number of viable microorganisms at a wound site. The buckwheat honey/povidone-iodine formulations or compositions are applied to a wound or skin condition, for example, in a liquid (e.g., by irrigating or lavaging the wound or skin condition with the liquid) or in a gel or an ointment. Liquid compositions provide immediate availability of the ions and povidone-iodine to the healing tissue. In contrast, gels or ointments provide regulated delivery of the ions, medicinal honey, and povidone-iodine to the healing tissue over a sustained period of time. In some embodiments, the buckwheat honey/povidone-iodine formulation or composition is applied to a wound dressing substrate, which is subsequently applied to the wound. Advantageously, a wound dressing including any of the compositions is contacted with the wound until it is healed (3-8 months) with wound dressing changes every 24-96 hours, thereby providing a moist environment enriched with fortified medicinal honey to facilitate healing of the skin. Similarly, a dressing comprising any of the formulations described herein can be contacted with a skin condition over a period of time with periodic changes.

In another aspect of the invention, a buckwheat honey/povidine-iodine formulation or composition can be provided in a tube or spray.

In another aspect of the invention, a wound-healing or skin condition treatment bandage or dressing comprises a substrate comprising a reinforcing fiber substrate material and a wound-healing or skin condition treatment formulation comprised of buckwheat honey and povidone-iodine.

In another aspect of the invention, a wound-healing or skin condition treatment substrate comprises an effective amount of the wound-healing or skin condition treatment formulation.

In another aspect of the invention, in a wound-healing or skin condition treatment substrate, the substrate material comprises acetate, cotton (medical gauze), or alginate.

In another aspect of the invention, a wound-healing or skin condition treatment substrate comprises a 5″×4″ adhesive or non-adhesive bandage with from about 2 to about 8 grams of wound treatment dressing or formulation or composition.

In another aspect of the invention, a wound-healing or skin condition treatment substrate comprises about 4 grams of buckwheat honey/povidone-iodine formulation or composition.

In another aspect of the invention, in a wound-healing or skin condition treatment substrate, the buckwheat honey/povidone-iodine formulation or composition is impregnated into a reinforcing fiber substrate for application to a wound or skin condition and is released therefrom.

In another aspect of the invention, a method of treating a wound on or in a patient comprises directly applying an effective amount of a wound treatment formulation comprised of buckwheat honey and povidone-iodine.

In another aspect of the invention, a method of treating a skin condition on a patient comprises directly applying an effective amount of a skin condition treatment formulation comprised of buckwheat honey and povidone-iodine.

In another aspect of the invention, a method of treating a wound or a skin condition on a patient comprises contacting the wound or skin condition with a wound-healing or skin condition treatment dressing comprising a reinforcing fiber substrate material and a wound-healing or skin condition treatment formulation or composition comprised of buckwheat honey and povidone-iodine. The formulation could be, for example, as follows:

| | |
|---|---|
| (a) Buckwheat Honey | 2-8 grams, preferably 4 grams |
| (b) Povidone-iodine | 1-5% w/v (preferably 2.5%) |
| (c) H₂O (sterile) | Dependent on w/v dilution |

The buckwheat honey/povidone-iodine formulation is preferably applied to an entire wound or skin condition. The treatment protocol includes complete evaluation of the wound or skin condition and clinical preparation of the wound or skin condition. For example:

### Dressing Preparation:

An amount of 0.10 grams of povidone-iodine powder is dissolved in 1.0 cc sterile water, and the resulting solution is mixed with 4 grams buckwheat honey pure, strained. The mixture is impregnated into a 5″×4″ acetate, non-woven medical grade substrate. Multiples of the buckwheat/povidone-iodine solution are made for multiple dressings. Each such substrate is protected with polyethylene liners applied to both sides, and each impregnated substrate is sealed individually in a foil pouch constructed of white polyester film fused to aluminum foil that constitutes the entire dressing package. The dressings are sterilized using a gamma radiation (minimum 25 kGy) and verified as sterile before use.

### Clinical Procedure:

1. Clinical examination of patient's skin and condition of wound, including location, etiology, size, depth, viability of all tissues, odor, infection, culture, neurovascular status, and nutrition.

2. Debridement of all devitalize/necrotic, purulent tissue from the wound, using clinical judgment.

3. Wound irrigation.

4. Application of buckwheat honey/povidone-iodine dressing for complete coverage of wound, daily dressing, and appropriate and timely debridement/irrigation of wound (no soaking).

### EXAMPLES

Objects and advantages of this invention are further illustrated by the following examples, but the particular materials and amounts thereof recited in these examples, as well as other conditions and details, should not be construed to unduly limit this invention.

### Example 1

A composition comprises a buckwheat honey with high peroxide-induced and polyphenol-facilitated antimicrobial activity and an effective amount of povidone-iodine (1-5% w/v) to add non-peroxide antimicrobial activity, wherein each of the antimicrobial activities provides a different mechanism of antibacterial inhibition. This provides a broad spectrum antimicrobial activity effective to reduce the number of viable microorganisms at a wound site.

### Example 2

In a composition similar to the composition described in Example 1, the honey is monofloral buckwheat honey that naturally generates 2-4 mM hydrogen peroxide and which naturally contains 275-575 gallic acid equivalents of polyphenol compounds per gram of honey, and wherein the amount of povidone iodine 1-5% w/v per gram of honey.

### Example 3

A wound dressing comprises a composition of Example 1 or 2 and a support. The support is, for example, a hydrogel, a foam, a film, a hydrocolloid, an alginate, collagen, or a combination of any two or more thereof.

### Example 4

Prior to treatment, a patient should be thoroughly informed of the nature of the treatment and any risks, and the patient should sign appropriate consent forms. In addition, a complete medical history and physical examination should be carried out prior to the initiation of treatment, medical or surgical. Understanding the etiology of the wound and the factors that are affecting the wound healing process (i.e., weight bearing, vascular status, neuropathy, osteomyelitis, etc.) are as important. Obviously, addressing such factors as these is extremely important in the successful treatment with all types of skin conditions and wounds. Multiple surgical procedures may be necessary to remove the devitalized, necrotic tissue and the surface exudate (biofilm) that was present. Also, irrigation of the wounds can be performed at the same time the wounds are surgically debrided during hospitalization.

A combined buckwheat honey and povidone-iodine sterile dressing or bandage can be used as an outpatient dressing. This dressing or bandage should be changed daily, and each patient should be seen in the office once a week. At each visit the patient would be examined, wounds would be measured, photographs would be taken, and wounds would be debrided, as necessary, with sharp non-excisional technique.

The sterile buckwheat honey (4 grams) and povidone iodine dressing or bandage (2.5% w/v) would be cut to the size and shape to completely cover a wound at each dressing change. The dressing or bandage would be applied directly to the surface of a wound entirely covering the wound. The dressing or bandage would be covered with at least one or more 4″×4″ fibrous (cotton) gauze and wrapped with 4″ fibrous (cotton) KLING® gauze dressings. Preferably the entire wound is covered at every dressing change in this manner whether in the office or at home. The patient's spouse or home health care provider should be educated on the proper technique for the dressing changes. The dressings should be kept clean and dry.

There could be indications where twice daily dressing changes are required. These indications could include increased wound drainage or increased wound desiccation.

While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

### REFERENCES

1. Zumla A, Lulat A. Honey-A remedy rediscovered. J R Soc Med 1989; 82:384-5.
2. Molan P C Potential of Honey in treatment of wounds and burns. Am J Clin Dermatol 2001; 2:13-9.
3. Brudzynski, Katrina, Kamal Abubaker, Tony Wang Powerful bacterial killing by buckwheat honeys is concentration-dependent, involves complete DNA degradation and requires hydrogen peroxide. Frontiers in MICROBIOLOGY July 2012; Vol 3, Article 242.
4. Armon P J (1980) The use of honey in the treatment of infected wounds. Trop Doct 10:91PubMed.
5. Molan P C (2006) The evidence supporting the use of honey as a wound dressing. Int J Low Extrem 5:40
6. Effem S E (1988) Clinical observations on the wound healing properties of honey. Br J Surg 75:679-681
7. Molan P C, Betts J A (2004) Clinical usage of honey as a wound dressing: an update. J Wound Care 13(9):353-356PubMedCrossRefGoogle Scholar
8. White J W (1975) Composition of honey. In: Crane E (ed) Honey: a comprehensive survey. Heinemann, London, pp 157-206
9. Luciana P, Fernandez S L (2005) Venous ulcer: epidemiology, physiopathology, diagnosis and treatment. Int Soc Dermatol 44:449-456.
10. Yapucu Güneş U, Eşer I (2007) Effectiveness of a honey dressing for healing pressure ulcers. J Wound Ostomy Cont Nurs 34(2):184-190
11. Okeniyi J A, Olubanjo O O, Ogunlesi T A, Oyelami O A (2005) Comparison of healing of incised abscess wounds with honey and EUSOL dressing. J Alternative Compl Med 11(3):511-513
12. Subrahmanyam M (1998) A prospective randomized clinical and histological study of superficial burn wound healing with honey and silver sulfadiazine. Burns 24(2):157-161PubMed
13. Wood B, Rademaker M, Molan P C (1997) Manuka honey, a low cost leg ulcer dressing. N Z Med J 110(1040):107
14. Subrahmanyam M (1994) Honey-impregnated gauze versus amniotic membrane in the treatment of burns. Burns 20(4):331-333PubMedCrossRefGoogle Scholar
15. Ndayisaba G, Bazira L, Habonimana E, Muteganya D (1993) Clinical and bacteriological results in wounds treated with honey. J Orthop Surg 7(2):202-204 Google Scholar
16. Weheida S M, Nagubi H H, El-Banna H M, Marzouk S (1991) Comparing the effects of two dressing techniques on healing of low grade pressure ulcers. J Med Res Inst 12(2):259-278
17. Kramer S A (1999) Effect of povidone-iodine on wound healing: a review. J Vasc Nurs 17(1):17-23 PubMed
18. Efem S E E (1988) Clinical observations on the wound healing properties of honey. Br J Surg 75:679-681 PubMed
19. Hamdy M H, El Banby M A, Khakifa K I, Gad E M (1988) The antimicrobial effect of honey in the management of septic wounds. Proceedings of the Fourth International Conference on Apiculture in Tropical Climates, Cairo, Egypt 6-10 November London, UK; International Bee Research Association
20. Mphande A N, Killowe C, Phalira S, Jones H W, Harrison W J (2007) Effects of honey and sugar dressings on wound healing. J Wound Care 16(7):317-319 PubMed
21. Subrahmanyam M (1991) Topical application of honey in treatment of burns. Br J Surg 78(4):497-498 PubMed
22. Medhi B, Puri A, Upadhyay S, Kaman L (2008) Topical application of honey in the treatment of wound healing: a metaanalysis. Alternative Med 10(4):166-169
23. U.S. Pat. No. 5,652,274, THERAPEUTIC-WOUND HEALING COMPOSITIONS AND METHODS FOR PREPARING AND USING SAME, Alain Martin, Jul. 29, 1997
24. Efem S E. Clinical observations on the wound healing properties of honey. Br J Surg. 1988; 75:679-81. doi: 10.1002/bjs.1800750718. [PubMed]
25. Postmes T, van den Bogaard A E, Hazen M. Honey for wounds, ulcers, and skin graft preservation. Lancet. 1993; 341:756-7. doi: 10.1016/0140-6736(93)90527-N. [PubMed]
26. Ranzato E, Martinotti S, Volante A, Mazzucco L, Burlando B. Platelet lysate modulates MMP-2 and MMP-9 expression, matrix deposition and cell-to-matrix adhesion in keratinocytes and fibroblasts. Exp Dermatol. 2011; 20:308-13. doi: 10.1111/j.1600-0625.2010.01173.x. [PubMed]
27. Gurtner G C, Werner S, Barrandon Y, Longaker M T. Wound repair and regeneration. Nature. 2008; 453:314-21. doi: 10.1038/nature07039. [PubMed]
28. Molan P C. Potential of honey in the treatment of wounds and burns. Am J Clin Dermatol. 2001; 2:13-9. doi: 10.2165/00128071-200102010-00003. [PubMed]
29. Ranzato E, Martinotti S, Burlando B. Epithelial mesenchymal transition traits in honey-driven keratinocyte wound healing: Comparison among different honeys. Wound Repair Regen. 2012; 20:778-85. doi: 10.1111/j.1524-475X.2012.00825.x. [PubMed]
30. Ranzato E, Mazzucco L, Patrone M, Burlando B. Platelet lysate promotes in vitro wound scratch closure of human dermal fibroblasts: Different roles of cell calcium, P38, ERK and PI3K/AKT. J Cell Mol Med. 2009; 13:2030-8. doi: 10.1111/j.1582-4934.2008.00467.x. [PubMed]
31. Ranzato E, Patrone M, Pedrazzi M, Burlando B. Hmgb1 promotes wound healing of 3T3 mouse fibroblasts via RAGE-dependent ERK1/2 activation. Cell Biochem Biophys. 2010; 57:9-17. doi: 10.1007/s12013-010-9077-0. [PubMed]
32. Houghton P J, Hylands P J, Mensah A Y, Hensel A, Deters A M. In vitro tests and ethnopharmacological investigations: Wound healing as an example. J Ethnopharmacol. 2005; 100:100-7. doi: 10.1016/j.jep.2005.07.001. [PubMed]
33. Iftikhar F, Arshad M, Rasheed F, Amraiz D, Anwar P, Gulfraz M. Effects of acacia honey on wound healing in various rat models. Phytother Res. 2010; 24:583-6. [PubMed]
34. Lusby P E, Coombes A, Wilkinson J M. Honey: A potent agent for wound healing? J Wound Ostomy Continence Nurs. 2002; 29:295-300. [PubMed]
35. Gethin G, Cowman S. Case series of use of Manuka honey in leg ulceration. Int Wound J. 2005; 2:10-5. doi: 10.1111/j.1742-4801.2005.00078.x. [PubMed]
36. Ranzato E, Burlando B. Signaling pathways in wound repair. In: Middleton J E, editor. Wound healing: Process, phases and promotion. Hauppauge, N.Y.: Nova Publishers Inc.; 2011. pp. 123-35.
37. Cipriani V, Ranzato E, Balbo V, Mazzucco L, Cavaletto M, Patrone M. Long-term effect of platelet lysate on primary fibroblasts highlighted with a proteomic approach. J Tissue Eng Regen Med. 2009; 3:531-8. doi: 10.1002/term.195. [PubMed]
38. Ranzato E, Balbo V, Boccafoschi F, Mazzucco L, Burlando B. Scratch wound closure of C2C12 mouse myoblasts is enhanced by human platelet lysate. Cell Biol Int. 2009; 33:911-7. doi: 10.1016/j.cellbi.2009.06.017. [PubMed]
39. Ranzato E, Boccafoschi F, Mazzucco L, Patrone M, Burlando B. Role of ERK1/2 in platelet lysate-driven endothelial cell repair. J Cell Biochem. 2010; 110:783-93. doi: 10.1002/jcb.22591. [PubMed]
40. Ranzato E, Martinotti S, Magnelli V, Murer B, Biffo S, Mutti L, et al. Epigallocatechin-3-gallate induces mesothelioma cell death via H(2) O(2)-dependent T-type Ca(2+) channel opening. J Cell Mol Med. 2012; 16:2667-78. doi: 10.1111/j.1582-4934.2012.01584.x. [PMC free article] [PubMed]
41. Ranzato E, Patrone M, Mazzucco L, Burlando B. Platelet lysate stimulates wound repair of HaCaT keratinocytes. Br J Dermatol. 2008; 159:537-45. [PubMed]
42. Ranzato E, Patrone M, Pedrazzi M, Burlando B. HMGb1 promotes scratch wound closure of HaCaT keratinocytes via ERK1/2 activation. Mol Cell Biochem. 2009; 332:199-205. doi: 10.1007/s11010-009-0192-4. [PubMed]
43. Ranzato E, Marinotti S, Pedrazzi M, Patrone M. High mobility group box protein-1 in wound repair. Cells. 2012; 1:699-710. doi: 10.3390/cells1040699. [PMC free article] [PubMed]
44. Li J, Kim Y N, Bertics P J. Platelet-derived growth factor-stimulated migration of murine fibroblasts is associated with epidermal growth factor receptor expression and tyrosine phosphorylation. J Biol Chem. 2000; 275:2951-8. doi: 10.1074/jbc.275.4.2951. [PubMed]

We claim:

1. A therapeutic wound-healing or skin condition treatment composition comprising a wound-healing or skin condition treatment effective amount of buckwheat honey and one or more bacterial agents

2. The therapeutic wound-healing or skin condition treatment composition of claim 1, wherein the buckwheat honey is 100% pure and strained.

3. The therapeutic wound-healing or skin condition treatment composition of claim 1 which comprises povidone-iodine as a bacterial agent.

4. The therapeutic wound-healing or skin condition treatment composition of claim 3, wherein the povidone-iodine comprises an about 9 to about 12% by volume iodine solution mixed with sterile water to dilute it to from about 1.5 to about 4.0% by volume.

5. The therapeutic wound-healing or skin condition treatment composition of claim 4, wherein the povidone-iodine solution is diluted to about 2.5%.

6. The therapeutic wound-healing or skin condition treatment composition of claim 3, wherein the povidone-iodine is in the form of a white powder.

7. The therapeutic wound-healing or skin condition treatment composition of claim 1 which can be provided in a tube or spray.

8. The therapeutic wound-healing or skin condition treatment composition of claim 1 which includes a gelling agent.

9. The therapeutic wound-healing or skin condition treatment composition of claim 8, wherein the gelling agent is sufficient to thicken the mixture and to substantially prevent the composition from running and to release a desired amount of the dressing to the wound.

10. A therapeutic wound-healing or skin condition treatment bandage or dressing comprising substrate material and a therapeutic wound-healing or skin treatment condition composition of claim 1.

11. The therapeutic wound-healing or skin condition treatment bandage or dressing of claim 10 which comprises a wound-healing or skin condition treatment effective amount of the therapeutic wound-healing or skin condition treatment composition.

12. The therapeutic wound-healing or skin condition treatment bandage or dressing of claim 10, wherein the substrate material comprises acetate, cotton (medical gauze), or alginate.

13. The therapeutic wound-healing or skin condition treatment bandage or dressing of claim 12 which comprises a 5″×4″ adhesive and non-adhesive bandage with from about 2 to 6 grams of wound healing composition.

14. The therapeutic wound-healing or skin condition treatment bandage or dressing of claim 13 which comprises about 4 grams of wound-healing or skin condition treatment composition.

15. The therapeutic wound-healing or skin condition treatment bandage or dressing of claim 10, wherein the wound-healing or skin condition treatment composition is impregnated into a reinforcing fiber substrate for application to a wound and is released therefrom.

16. A method of therapeutically treating a wound on or in a patient which comprises directly applying an effective amount of a wound-healing composition of claim 1 to the wound.

17. A method of therapeutically treating a skin condition on a patient which comprises directly applying an effective amount of a skin condition treatment composition of claim 1 to the wound.

18. A method of therapeutically treating a wound on a patient which comprises contacting the wound with a therapeutic wound-healing bandage or dressing of claim 10.

19. A method of therapeutically treating a skin condition on a patient which comprises contacting the skin condition with a therapeutic skin condition treatment bandage or dressing of claim 10.
